(19) United States
(12) Patent Application Publication
(10) Pub. No.: US 2012/0022018 A1
(43) Pub. Date: Jan. 26, 2012

(54) ANTI-VIRAL PROPERTIES OF ALOE VERA AND ACQUIRED IMMUNE DEFICIENCY SYNDROME (AIDS) TREATMENT

(75) Inventor: Ivan E. Danhof, Grand Prairie, TX (US)

(73) Assignee: NORTH TEXAS MEDICAL ASSOCIATES, Grand Prairie, TX (US)

(21) Appl. No.: 13/187,222

(22) Filed: Jul. 20, 2011

Related U.S. Application Data
(60) Provisional application No. 61/367,358, filed on Jul. 23, 2010.

(51) Int. Cl. A61K 31/715; A61P 37/00; A61P 31/18; A61K 31/736

(52) U.S. Cl. 514/54

(57) ABSTRACT

A pharmaceutical composition comprising a combination of formulations derived from Aloe vera for the treatment of Acquired Immune Deficiency Syndrome (AIDS) or HIV infection is described herein. The composition comprises: (i) an injectable sterile polymannan extract, (ii) Raidox (aloe anthraquinones and their diacetyl derivatives), and (iii) a freeze dried aloe vera powder, aloe vera juice, aloe gel or a combination. In addition one or more nutritional supplements comprising fatty acids, proteins, minerals and metals, vitamins, salts, amino acids, and other pharmaceutically acceptable excipients may also be include to counteract the chronic diarrhea, digestive upsets, and weight loss seen in some patients before and during the treatment course. A method for treating the AIDS or HIV infection using the composition of the present invention is also disclosed.

### CROSS-REFERENCE TO RELATED APPLICATIONS

This patent application is a non-provisional application of U.S. Provisional Patent Application No. 61/367,358 filed on Jul. 23, 2010 and entitled “Anti-Viral Properties of Aloe Vera and Acquired Immune Deficiency Syndrome (AIDS) Treatment” which is hereby incorporated by reference in its entirety.

### TECHNICAL FIELD OF THE INVENTION

The present invention relates in general to the field of Acquired Immune Deficiency Syndrome (AIDS) treatment, and more particularly, to compositions and uses of aloe vera, related products, and derivatives for the treatment of AIDS.

### STATEMENT OF FEDERALLY FUNDED RESEARCH

None.

### REFERENCE TO A SEQUENCE LISTING

None.

### BACKGROUND OF THE INVENTION

Without limiting the scope of the invention, its background is described in connection with anti-viral properties of aloe vera and related compounds and therapeutic uses of the same.

U.S. Pat. No. 7,169,414 issued to Shupe and Coats (2007) describes antimicrobial agents and method for isolation thereof from the gel liquid of Aloe vera includes at least one antimicrobial agent isolated from the clear gel isolated from the whole leaf of the Aloe vera plant, wherein the antimicrobial agent is an agent produced by the Aloe vera and/or indigenous bacteria that colonize the Aloe vera plant, is disclosed.

WIPO Patent No. WO/1993/008810 issued to Mcanalley et al. (1993) discloses the use of Acemannan in treating a number of conditions where the principal mechanism of resolution or cure requires intervention by the patient's immune system. Acemannan has direct stimulatory effects on the immune system. Methods for treating cancer, viral diseases, respiratory and immune regulatory diseases, inflammations, infections and infestations by administering an acetylated mannan derivative, such as acemannan derived from aloe, are described. The method finds use in tissue cultures, animals and plants.

### SUMMARY OF THE INVENTION

The present invention is a composition and method of treatment for acquired immune deficiency syndrome (AIDS). The composition disclosed herein comprises a combination of polymannan extract (PME), anthraquinones, and freeze dried aloe powder along with one or more nutritional supplements for the treatment of AIDS.

One embodiment of the instant invention discloses a pharmaceutical composition for treating, amelioration of symptoms or both of an Acquired Immune Deficiency Syndrome (AIDS) or a HIV infection in a subject comprising: a sterile injectable polymannan extract formulation comprising one or more aloe polysaccharides, wherein the aloe polysaccharides comprise one or more small chain, medium chain, large chain, very-large chain polysaccharides, or any combinations thereof, a formulation comprising one or more aloe anthraquinones, diacetyl anthraquinone derivatives, and combinations and modifications thereof, a formulation comprising a freeze dried aloe powder, an aloe juice, an aloe gel or a combination wherein the powder is administered as is or after reconstitution in a suitable liquid, and a formulation comprising one or more optional nutritional supplements selected from the group consisting of fatty acids, proteins, minerals and metals, vitamins, salts, amino acids, and other pharmaceutically acceptable excipients. In one aspect of the instant invention the polymannan extract is administered intravenously, intramuscularly, subcutaneously, intraperitoneally or by any other suitable parenteral route. In a specific aspect the polymannan extract is administered intravenously at a dose of 10-15 mg three times a week. In a related aspect the formulations other than the polymannan extract are administered orally. In another aspect the one or more aloe polysaccharides in the polymannan extract have molecular weights ranging from 50,000-10,000,000 Daltons.

The one or more aloe anthraquinones used in the composition of the present invention are selected from the group consisting of Aloe-emodin, 1,8-Dihydroxy-3-(hydroxymethyl-9,10-anthracenedione, 1,8-Dihydroxy-3-(hydroxymethyl)anthraquinone, 3-Hydroxymethylchrysazin, Aloe-Emodine-Anthranol, Aloinoside-A, Aloinoside-B, Aloin-A, 10-Glucopyranosyl-1,S-dihydroxy-3-(hydroxymethyl)-9(10)-anthracene, 1,8-Dihydroxy-3-hydroxymethyl-10-(6-hydroxymethyl)-3,4,5-trihydroxy-2-pyranosyl, anthrone, 10-{1′, 5′-anhydroglucosyl)-aloe-emodin-9-anthrone, Barbaloin, Aloin-B Epimere of Aloin-A, Isobarbaloin, Aloinoslde-A, Aloinoside-B, Anthranols, Anthraquinone-glycocide, Chrysaminic Acid, Chrysophanic Acid, ChrysoDhanol, Chrysophanol-glycoside, 1,8-Dihydroxyanthracene, Emodin, 1,3,8-Trihydroxy-6-methyl-9,10-anthracene-dione, 1,3,8-Trihydroxy-6-methylanthraquinone, 4,5,7-Trihydroxy-2-methylanthraquinone, Frangula emodin, Homonataloin, Hydroxymethylanthraquinone, Rhein, 9,10-4,5-Dihydroxy-9,10-dioxo-2-anthracene-carboxylic acid, 1,5-Dihydroxyanthraquinone-3-carboxylic acid, 4,5-Dihydroxyanthraquanone-2-carboxylic acid, Chrysazin-3-carboxylic acid, Trihydroxymethylanthraquinone, and modifications and derivatives thereof.

In another aspect the aloe anthraquinones are administered at a dose ranging from 50-75 mg/day. In yet another aspect the freeze dried aloe powder, the aloe juice, the aloe gel or the combination is administered at a dose of 400 mg one or more times a day. In one aspect the aloe juice comprises of about 2% total solids. In another aspect aloe juice comprises glucomannan polysaccharides. In a specific aspect the glucomannan polysaccharides have molecular weights ranging from 50,000-10,000,000 Daltons. In yet another aspect wherein the one or more fatty acids are selected from the group consisting of Linoleic Acid (LA), Gamma Linoleic Acid (GLA), Eicosapentaneoic Acid (EPA), Docosapentaneoic Acid (DPA), Docosahexaenoic Acid (DHA), and D-alpha-tocopherol. The composition as described hereinabove releases one or more cytocommunicators selected from the group consisting of TNF-α, IL-1β, IL-2, IL-6, and INF-γ, wherein the release stimulates the growth and cytolytic action of one or more Natural Killer (NK) cells in the subject.

In another aspect the present invention provides a method of treating, ameliorating symptoms or both of an Acquired Immune Deficiency Syndrome (AIDS) or a HIV infection in a subject comprising the steps of: identifying the subject in need of the treatment or amelioration of the symptoms against the AIDS or the HIV infection and administering a therapeutically effective amount of a pharmaceutical composition sufficient to treat or ameliorate the symptoms of the AIDS or the HIV infection.

The pharmaceutical composition used in the method of the present invention comprises the following:

(i) a sterile injectable polymannan extract formulation comprising one or more aloe polysaccharides, wherein the aloe polysaccharides comprise one or more small chain, medium chain, large chain, very-large chain polysaccharides, or any combinations thereof,
(ii) a formulation comprising one or more aloe anthraquinones, diacetyl anthraquinone derivatives, and combinations and modifications thereof,
(iii) a formulation comprising a freeze dried aloe powder, an aloe juice, an aloe gel or a combination wherein the powder is administered as is or after reconstitution in a suitable liquid, and
(iv) a formulation comprising one or more optional nutritional supplements selected from the group consisting of fatty acids, proteins, minerals and metals, vitamins, salts, amino acids, and other pharmaceutically acceptable excipients.

In one aspect the polymannan extract is administered intravenously. The dosage of the injected polymannan extract is 10-15 mg three times a week. The aloe anthraquinones the freeze dried aloe powder and the optional nutritional supplements are administered orally. The one or more aloe anthraquinones used herein are selected from the group consisting of Aloe-emodin, 1,8-Dihydroxy-3-(hydroxymethyl-9,10-anthracenedione, 1,8-Dihydroxy-3-(hydroxymethyl)anthraquinone, 3-Hydroxymethylchrysazin, Aloe-Emodine-Anthranol, Aloinoside-A, Aloinoside-B, Aloin-A, 10-Glucopyranosyl-1,S-dihydroxy-3-(hydroxymethyl)-9(10)-anthracene, 1,8-Dihydroxy-3-hydroxymethyl-10-(6-hydroxymethyl)-3,4,5-trihydroxy-2-pyranosyl, anthrone, 10-{1′,5′-anhydroglucosyl)-aloe-emodin-9-anthrone, Barbaloin, Aloin-B Epimere of Aloin-A, Isobarbaloin, Aloinoslde-A, Aloinoside-B, Anthranols, Anthraquinone-glycocide, Chrysaminic Acid, Chrysophanic Acid, ChrysoDhanol, Chrysophanol-glycoside, 1,8-Dihydroxyanthracene, Emodin, 1,3,8-Trihydroxy-6-methyl-9,10-anthracene-dione, 1,3,8-Trihydroxy-6-methylanthraquinone, 4,5,7-Trihydroxy-2-methylanthraquinone, Frangula emodin, Homonataloin, Hydroxymethylanthraquinone, Rhein, 9,10-4,5-Dihydroxy-9,10-dioxo-2-anthracene-carboxylic acid, 1,5-Dihydroxyanthraquinone-3-carboxylic acid, 4,5-Dihydroxyanthraquanone-2-carboxylic acid, Chrysazin-3-carboxylic acid, Trihydroxymethylanthraquinone, and modifications and derivatives thereof. In a specific aspect the aloe anthraquinones are administered at a dose ranging from 50-75 mg/day. In another aspect the aloe juice comprises glucomannan polysaccharides. In yet another aspect the freeze dried aloe powder, the aloe juice, the aloe gel or the combination is administered at a dose of 400 mg one or more times a day.

The one or more fatty acids in the nutritional supplements are selected from the group consisting of Linoleic Acid (LA), Gamma Linoleic Acid (GLA), Eicosapentaneoic Acid (EPA), Docosapentaneoic Acid (DPA), Docosahexaenoic CAid (DHA), and D-alpha-tocopherol. In another aspect the composition releases one or more cytocommunicators selected from the group consisting of TNF-α, IL-1β, IL-2, IL-6, and INF-γ, wherein the release stimulates the growth and cytolytic action of one or more Natural Killer (NK) cells in the subject.

Yet another embodiment of the instant invention discloses a pharmaceutical composition for treating, amelioration of symptoms or both of an Acquired Immune Deficiency Syndrome (AIDS) or a HIV infection in a subject comprising: a sterile injectable polymannan extract formulation comprising one or more aloe polysaccharides, wherein the aloe polysaccharides comprise one or more small chain, medium chain, large chain, very-large chain polysaccharides, or any combinations thereof, a formulation comprising one or more aloe anthraquinones, diacetyl anthraquinone derivatives, and combinations and modifications thereof, a formulation comprising a freeze dried aloe powder, an aloe juice, an aloe gel or a combination wherein the powder is administered as is or after reconstitution in a suitable liquid and a formulation of a nutritional supplement comprising:

| | |
|---|---|
| Linoleic Acid (LA) | 280 mg |
| Gamma Linolenic Acid (GLA) | 80 mg |
| Eicosapentaenoic Acid (EPA) | 45 mg |
| Doscosapentaenoic Acid (DPA) | 9 mg |
| Docosahexaenoic Acid (DHA) | 30 mg |
| d'alpha tocopherol | 15 mg |

In one aspect the polymannan extract is administered intravenously, whereas the formulations other than the polymannan extract are administered orally. In another aspect the one or more aloe anthraquinones are selected from the group consisting of Aloe-emodin, 1,8-Dihydroxy-3-(hydroxymethyl-9,10-anthracenedione, 1,8-Dihydroxy-3-(hydroxymethyl)anthraquinone, 3-Hydroxymethylchrysazin, Aloe-Emodine-Anthranol, Aloinoside-A, Aloinoside-B, Aloin-A, 10-Glucopyranosyl-1,S-dihydroxy-3-(hydroxymethyl)-9(10)-anthracene, 1,8-Dihydroxy-3-hydroxymethyl-10-(6-hydroxymethyl)-3,4,5-trihydroxy-2-pyranosyl, anthrone, 10-{1′,5′-anhydroglucosyl)-aloe-emodin-9-anthrone, Barbaloin, Aloin-B Epimere of Aloin-A, Isobarbaloin, Aloinoslde-A, Aloinoside-B, Anthranols, Anthraquinone-glycocide, Chrysaminic Acid, Chrysophanic Acid, ChrysoDhanol, Chrysophanol-glycoside, 1,8-Dihydroxyanthracene, Emodin, 1,3,8-Trihydroxy-6-methyl-9,10-anthracene-dione, 1,3,8-Trihydroxy-6-methylanthraquinone, 4,5,7-Trihydroxy-2-methylanthraquinone, Frangula emodin, Homonataloin, Hydroxymethylanthraquinone, Rhein, 9,10-4,5-Dihydroxy-9,10-dioxo-2-anthracene-carboxylic acid, 1,5-Dihydroxyanthraquinone-3-carboxylic acid, 4,5-Dihydroxyanthraquanone-2-carboxylic acid, Chrysazin-3-carboxylic acid, Trihydroxymethylanthraquinone, and modifications and derivatives thereof. In yet another aspect the aloe juice comprises glucomannan polysaccharides.

A method of treating, ameliorating symptoms or both of an Acquired Immune Deficiency Syndrome (AIDS) or a HIV infection in a subject is described in an embodiment of the present invention, wherein the method comprises the steps of: identifying the subject in need of the treatment or amelioration of the symptoms against the AIDS or the HIV infection and administering a therapeutically effective amount of a pharmaceutical composition sufficient to treat or ameliorate the symptoms of the AIDS or the HIV infection, wherein the pharmaceutical composition comprises: (i) a sterile injectable polymannan extract formulation comprising one or more aloe polysaccharides, wherein the aloe polysaccharides comprise one or more small chain, medium chain, large chain, very-large chain polysaccharides, or any combinations thereof, (ii) a formulation comprising one or more aloe anthraquinones, diacetyl anthraquinone derivatives, and combinations and modifications thereof, (iii) a formulation comprising a freeze dried aloe powder, an aloe juice, an aloe gel or a combination wherein the powder is administered as is or after reconstitution in a suitable liquid, and (iv) a formulation of a nutritional supplement comprising:

| | |
|---|---|
| Linoleic Acid (LA) | 280 mg |
| Gamma Linolenic Acid (GLA) | 80 mg |
| Eicosapentaenoic Acid (EPA) | 45 mg |
| Doscosapentaenoic Acid (DPA) | 9 mg |
| Docosahexaenoic Acid (DHA) | 30 mg |
| d'alpha tocopherol | 15 mg |

In one aspect the polymannan extract is administered intravenously at a dose of 10-15 mg three times a week and the other formulations are administered orally. In another aspect the aloe anthraquinones are administered at a dose ranging from 50-75 mg/day and the freeze dried aloe powder, the aloe juice, the aloe gel or the combination is administered at a dose of 400 mg one or more times a day. In a related aspect the composition releases one or more cytocommunicators selected from the group consisting of TNF-α, IL-13, IL-2, IL-6, and INF-γ, wherein the release stimulates the growth and cytolytic action of one or more Natural Killer (NK) cells in the subject.

In another embodiment the pharmaceutical composition for treating, amelioration of symptoms or both of an Acquired Immune Deficiency Syndrome (AIDS) or a HIV infection in a subject comprising: a sterile injectable polymannan extract formulation comprising one or more aloe polysaccharides, wherein the aloe polysaccharides comprise one or more small chain, medium chain, large chain, very-large chain polysaccharides, or any combinations thereof, a formulation comprising one or more aloe anthraquinones, diacetyl anthraquinone derivatives, and combinations and modifications thereof, a formulation comprising a freeze dried aloe powder, an aloe juice, an aloe gel or a combination wherein the powder is administered as is or after reconstitution in a suitable liquid, and a formulation of a nutritional supplement comprising:

Nutritional Information per serving

| | | | | | |
|---|---|---|---|---|---|
| Serving Size | 48 | grams | Fat | 1 | gm |

| Calories | 160 | | *Cholesterol | 0 | gm |
|---|---|---|---|---|---|
| Protein | 13 | gms | Sodium | 120 | mg |
| Carbohydrates | 26 | gms | Potassium | 660 | mg |

Percentages of U.S. Recommended Daily Allowances (U.S. RDA) per serving:

| Protein | 30% | Vitamin E | 50% | Copper | 50% | |
|---|---|---|---|---|---|---|
| Vitamin A | 70% | Vitamin B₆ | 50% | Biotin | 50% | |
| Vitamin C | 50% | Folic Acid | 50% | Pantothenic Acid | 50% | |
| Thiamine | 50% | Vitamin B₁₂ | 50% | **Chromium | 23 | mcg |
| Riboflavin | 60% | Phosphorus | 20% | **Selenium | 23 | mcg |
| Niacin | 50% | Iodine | 50% | **Manganese | 2 | mg |
| Calcium | 25% | Magnesium | 50% | **Fiber | 3 | gms |
| Iron | 50% | Zinc | 50% | **Octacosanol | 2000 | mcg |
| Vitamin D | 50% | | | | | |

Amino Acids per 48 gram serving

| | | | | | | | | |
|---|---|---|---|---|---|---|---|---|
| Alanine | 375 | mg | Histidine | 320 | mg | Proline | 1425 | mg |
| Arginine | 450 | mg | Isoleucine | 655 | mg | Serine | 700 | mg |
| Aspartic acid | 850 | mg | ***Leucine | 1115 | mg | ***Threonine | 510 | mg |
| Cystine | 65 | mg | ***Lysine | 920 | mg | ***Tryptophan | 145 | mg |
| Glutamic acid | 2640 | mg | ***Methionine | 310 | mg | Tyrosine | 630 | mg |
| Glycine | 250 | mg | ***Phenylalanine | 600 | mg | ***Valine | 755 | mg |

Ingredients:
Fructose, Nonfat Milk Solids, Calcium Sodium Caseinate, Natural and Artificial flavors, Cellulose, Corn Bran, Potassium Chloride, Lecithin, Maltodextrin, Carrageenan, Wheat Germ Oil, (source of Octacosanol), Magnesium Oxide, Beta Carotene, Selenium, Ascorbic Acid, Ferrous Fumarate, d'Alpha Tocopherol Acetate, Chromium, Niacin, Aloe, Apple Pectin, Zinc Oxide, Manganese Sulfate, Vitamin A Palmitate, d-Calcium Pantothenate, Copper Sulfate, Pyridoxine Hydrochloride, Riboflavin, Thiamine Hydrochloride, Cobalamin Concentrate, Vitamin D₂, Folic Acid, Biotin and Potassium Chloride.

In one aspect the polymannan extract is administered intravenously and the formulations other than the polymannan extract are administered orally. In another aspect the one or more aloe anthraquinones are selected from the group consisting of Aloe-emodin, 1,8-Dihydroxy-3-(hydroxymethyl-9,10-anthracenedione, 1,8-Dihydroxy-3-(hydroxymethyl)anthraquinone, 3-Hydroxymethylchrysazin, Aloe-Emodine-Anthranol, Aloinoside-A, Aloinoside-B, Aloin-A, 10-Glucopyranosyl-1,S-dihydroxy-3-(hydroxymethyl)-9(10)-anthracene, 1,8-Dihydroxy-3-hydroxymethyl-10-(6-hydroxymethyl)-3,4,5-trihydroxy-2-pyranosyl, anthrone, 10-{1′,5′-anhydroglucosyl)-aloe-emodin-9-anthrone, Barbaloin, Aloin-B Epimere of Aloin-A, Isobarbaloin, Aloinoslde-A, Aloinoside-B, Anthranols, Anthraquinone-glycocide, Chrysaminic Acid, Chrysophanic Acid, ChrysoDhanol, Chrysophanol-glycoside, 1,8-Dihydroxyanthracene, Emodin, 1,3,8-Trihydroxy-6-methyl-9,10-anthracene-dione, 1,3,8-Trihydroxy-6-methylanthraquinone, 4,5,7-Trihydroxy-2-methylanthraquinone, Frangula emodin, Homonataloin, Hydroxymethylanthraquinone, Rhein, 9,10-4,5-Dihydroxy-9,10-dioxo-2-anthracene-carboxylic acid, 1,5-Dihydroxyanthraquinone-3-carboxylic acid, 4,5-Dihydroxyanthraquanone-2-carboxylic acid, Chrysazin-3-carboxylic acid, Trihydroxymethylanthraquinone, and modifications and derivatives thereof. In yet another aspect the aloe juice comprises glucomannan polysaccharides.

Another embodiment of the instant invention discloses a method of treating, ameliorating symptoms or both of an Acquired Immune Deficiency Syndrome (AIDS) or a HIV infection in a subject comprising the steps of: identifying the subject in need of the treatment or amelioration of the symptoms against the AIDS or the HIV infection and administering a therapeutically effective amount of a pharmaceutical composition sufficient to treat or ameliorate the symptoms of the AIDS or the HIV infection, wherein the pharmaceutical composition comprises: (a) a sterile injectable polymannan extract formulation comprising one or more aloe polysaccharides, wherein the aloe polysaccharides comprise one or more small chain, medium chain, large chain, very-large chain polysaccharides, or any combinations thereof, (b) a formulation comprising one or more aloe anthraquinones, diacetyl anthraquinone derivatives, and combinations and modifications thereof, (c) a formulation comprising a freeze dried aloe powder, an aloe juice, an aloe gel or a combination wherein the powder is administered as is or after reconstitution in a suitable liquid, and (d) a formulation of a nutritional supplement comprising:

Nutritional Information per serving

| Serving Size | 48 | grams | Fat | 1 | gm |
|---|---|---|---|---|---|
| Calories | 160 | | *Cholesterol | 0 | gm |
| Protein | 13 | gms | Sodium | 120 | mg |
| Carbohydrates | 26 | gms | Potassium | 660 | mg |

Percentages of U.S. Recommended Daily Allowances (U.S. RDA) per serving:

| Protein | 30% | Vitamin E | 50% | Copper | 50% | |
|---|---|---|---|---|---|---|
| Vitamin A | 70% | Vitamin B₆ | 50% | Biotin | 50% | |
| Vitamin C | 50% | Folic Acid | 50% | Pantothenic Acid | 50% | |
| Thiamine | 50% | Vitamin B₁₂ | 50% | **Chromium | 23 | mcg |
| Riboflavin | 60% | Phosphorus | 20% | **Selenium | 23 | mcg |
| Niacin | 50% | Iodine | 50% | **Manganese | 2 | mg |
| Calcium | 25% | Magnesium | 50% | **Fiber | 3 | gms |
| Iron | 50% | Zinc | 50% | **Octacosanol | 2000 | mcg |
| Vitamin D | 50% | | | | | |

Amine Acids per 48 gram serving

| | | | | | | | | |
|---|---|---|---|---|---|---|---|---|
| Alanine | 375 | mg | Histidine | 320 | mg | Proline | 1425 | mg |
| Arginine | 450 | mg | Isoleucine | 655 | mg | Serine | 700 | mg |
| Aspartic acid | 850 | mg | ***Leucine | 1115 | mg | ***Threonine | 510 | mg |
| Cystine | 65 | mg | ***Lysine | 920 | mg | ***Tryptophan | 145 | mg |
| Glutamic acid | 2640 | mg | ***Methionine | 310 | mg | Tyrosine | 630 | mg |
| Glycine | 250 | mg | ***Phenylalanine | 600 | mg | ***Valine | 755 | mg |

Ingredients:
Fructose, Nonfat Milk Solids, Calcium Sodium Caseinate, Natural and Artificial flavors, Cellulose, Corn Bran, Potassium Chloride, Lecithin, Maltodextrin, Carrageenan, Wheat Germ Oil, (source of Octacosanol), Magnesium Oxide, Beta Carotene, Selenium, Ascorbic Acid, Ferrous Fumarate, d'Alpha Tocopherol Acetate, Chromium, Niacin, Aloe, Apple Pectin, Zinc Oxide, Manganese Sulfate, Vitamin A Palmitate, d-Calcium Pantothenate, Copper Sulfate, Pyridoxine Hydrochloride, Riboflavin, Thiamine Hydrochloride, Cobalamin Concentrate, Vitamin D₂, Folic Acid, Biotin and Potassium Chloride.

In one aspect the polymannan extract is administered intravenously at a dose of 10-15 mg three times a week. In another aspect the formulations other than the polymannan extract are administered orally. In yet another aspect the aloe anthraquinones are administered at a dose ranging from 50-75 mg/day and the freeze dried aloe powder, the aloe juice, the aloe gel or the combination is administered at a dose of 400 mg one or more times a day. In one aspect the composition releases one or more cytocommunicators selected from the group consisting of TNF-α, IL-1β, IL-2, IL-6, and INF-γ, wherein the release stimulates the growth and cytolytic action of one or more Natural Killer (NK) cells in the subject.

### BRIEF DESCRIPTION OF THE DRAWINGS

None.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “AIDS” refers to a status, as defined by the Center for Disease Control criteria. These criteria are any of the following two characteristics: 1. Infection with HIV and a CD4+cell count below 200/mm³ or a CD4+ cell count below 14%, with or without an opportunistic infection; or, 2. Infection with HIV and a CD4+ cell count greater than 200/mm 3 or CD+ cell count greater than 14% but who exhibit one or more of the following conditions: Candidiasis of bronchi, trachea or lungs, Candidiasis, esophageal, Cervical cancer, invasive, Coccidioid mycosis, disseminated or extrapulmonary , Cryptococcoses, extrapulmonary, Cryptosporidiosis, chronic intestinal (>1 month's duration), Cytomegalovirus disease (other than liver, spleen or nodes), Cytomegalus retinitis (with loss of vision), Encephalopathy, HIV-related, Herpes simplex: chronic ulcer(s) (>1 month's duration) or bronchitis, pneumonitis, or esophagitis, Histoplasmosis, disseminated or extrapulmonary, Isosoporiasis, chronic intestinal (>1 month's duration) , Kaposi's sarcoma, Lymphoma, immunoblastic (or equivalent term), Lymphoma, primary, or brain, MAIS complex or M. kansasii, disseminated or extrapulmonary, M. tuberculosis, any site (pulmonary or extrapulmonary), Pneumocystis carinii pneumonia, Pneumonia, recurrent, Progressive multifocal leukoencephalopathy, Salmonella septicemia, recurrent, Toxoplasmosis of brain, and Wasting syndrome due to HIV.

The term “aloe composition” as described in various embodiments of the instant invention refers to any extract or processed form of a plant of genus aloe, family Liliaceae. For example, aloe extracts and processed forms of aloe for use with the invention may be obtained from aloe arbrorescens, aloe barbandensis, or aloe ferox species of aloe. Any part of the plant may be processes or extracted, such as the leaf, stem or flower.

The term “pharmaceutically acceptable” is intended to include a carrier, a diluent or excipient that is compatible with the other ingredients of the formulation and not deleterious to the recipient thereof.

The terms “administration of” or “administering a” compound should be understood to indicate providing a compound of the invention to the individual in need of treatment in a form that can be introduced into that individual's body in a therapeutically useful form and therapeutically useful amount, including, but not limited to: oral dosage forms, such as tablets, capsules, syrups, suspensions, and the like; injectable dosage forms, such as IV, IM, or IP, and the like; transdermal dosage forms, including creams, jellies, powders, or patches; buccal dosage forms; inhalation powders, sprays, suspensions, and the like; and rectal suppositories.

The terms “effective amount” or “therapeutically effective amount” includes the amount of the subject compound that will elicit the biological or medical response of a tissue, system, animal or human that is being sought by the researcher, veterinarian, medical doctor or other clinician. As used herein, the term “treatment” refers to the treatment of the mentioned conditions, particularly in a patient who demonstrates symptoms of the disease or disorder.

As used herein, the term “treatment ” or “treating” includes any administration of a compound of the present invention and includes (1) inhibiting the disease in an animal that is experiencing or displaying the pathology or symptomatology of the diseased (i.e., arresting further development of the pathology and/or symptomatology), or (2) ameliorating the disease in an animal that is experiencing or displaying the pathology or symptomatology of the diseased (i.e., reversing the pathology and/or symptomatology). The term “controlling” includes preventing treating, eradicating, ameliorating or otherwise reducing the severity of the condition being controlled.

The present invention described compositions and methods for the treatment of Acquired Immune Deficiency Syndrome (AIDS), using a combination of formulations derived from Aloe vera. The composition of the present invention includes a sterile injectable polymannan extract, one or more aloe anthraquinones and their derivatives, and freeze dried aloe powder, aloe juice or an aloe gel. One or more nutritional supplements are also described that may be given to patients who experience chronic diarrhea and digestive upsets during the course of the treatment.

The earliest studies evaluating the effect of aloe preparations in AIDS patients were conducted by Terry L. Pulse, M.D., who, in the late 1980's conducted a study of 31 HIV-positive patients. Two subjects dropped out of the study, but the extended protocol (six-months) was completed on 29 subjects. The data were published'.

Based on the modified Walter Reed Score, the distribution of the subjects was: Full-blown AIDS: 15 Subjects, AIDS-Related Complex (ARC): 12 Subjects HIV Seropositive: 2 Subjects

The subjects were placed on 1200 mg of total solids in hand-filleted aloe vera juice (6-ounces) daily. In addition, dietary supplements consisting of essential fatty acids, and a protein-rich, vitamin-mineral powder were ingested daily. After six months, 27 of the 29 patients showed improvement on their Walter Reed Scores. All had improved Karnofsky Quality of life Scores. T4 cell counts, some originally determined to be less than 200, had climbed to the 400-500 range and a few even as high as 800. HPV P-24 Core antigen dropped with 25% of the reactive patients showing conversion to negative at 90 and 180 days.

Symptomatic Assessment at 6 months: Energy levels improved significantly within 3-5 days of treatment, fevers disappeared, night sweats ceased, coughs decreased significantly or stopped altogether, shortness-of-breath decreased very significantly so some patients were able to resume their gym workouts, which had been abrogated by their illness, palpable lymph nodes decreased in size, diarrhea stopped, strength and stamina improved, and reversal of disease-associated cachexia with a welcome increase in body weight

A number of patients with chronic diarrhea and/or respiratory symptoms, who were no longer employable owing to the severity of the symptoms, were able to resume their normal employment roles.

Clinical Assessment: No biochemical abnormalities on the computerized chemistry assessment, AZT-induced anemia was reversed on the aloe/nutritional support regimen, chest radiographs remained normal throughout the study, and showed improvement in a number of patients, and no changes in EKG were noted, significant reduction in reactivity of hypersensitivity skin testing at the end of 90 days.

The current generation of treatment modalities has performed satisfactorily in controlling viremia with significantly increased well-being and increased longevity of the patients, however the current treatments are unable to inhibit, in a significant manner, the intracellular replication cycle of the viral particles. The novel treatment approaches of the present invention have achieved some control improvement in inhibiting the replication of the virus intercellularly.

Polymannan Extract: comprises a very high molecular weight (2-10 million Daltons) molecules composed predominantly of mannose and glucose with very small amounts of arabinose and galactose. This carbohydrate fraction is produced under U.S. Pat. No. 6,083,508 (2000), and further modified by U.S. Provisional Patent No. 61/294,970 which maximizes the capture of a maximum quantity of the very large polysaccharide species. The polysaccharides are partially acetylated, which is a requisite constituent for their potent immunomodulatory activity upon intravenous administration. Upon injection these large molecules bind to mannose receptors on the monocyte/macrophage cells resulting in the elaboration of a suite of cytocommunicators including TNF-α, IL-13, IL-2, IL-6, IFN-γ. These entities restore to normal an impaired/defunct immune surveillance system, stimulate β lymphocytes (and antibody production), significantly increase the output and availability of natural killer cells, which, vivo, result in very significant anti-tumor and other salubrious activities.²⁻³

Raidox: comprises a mixture of several small molecular anthraquinone species derived from aloe. These water-soluble molecules are capable of entering the infected T4 cell where it partially disrupts the viral capsule, which abrogates the completion of the replication cycle. These activities have been substantiated in human T4 cell cultures. The material is administered orally. Limited safety studies in some AIDS patients showed no symptoms after oral ingestion of 5 times the recommended treatment dosage.

Freeze-dried Aloe Powder: comprises an increased quantity of the very large molecular weight carbohydrates, which combine with receptors on the small intestinal mucosal “M” cells. Once binding has taken place, the “M” cell uptake of opportunistic infectious agents is completely blocked. In addition the endocytotic absorption of the large molecular carbohydrates stimulates additional release of the cytocommunicators, the process of which is described herein.

Thus the three important advantages of the treatment system of the instant invention include: (i) significant increase in the number of T4 helper cells, (ii) intracellular interruption of HIV replication, and (iii) prevention of uptake of intestinal opportunistic infectious agents via “M” cell blockade.

Aloe Polysaccharides added to viral cultures induce viral envelope glycoprotein dysynthesis: Acemannan, an aloe polysaccharide (APS), having a molecular weight range of about 900,000 to 1,500,000 Daltons, was added to HIV-1 infected monocytes cultures and demonstrated that the APS induced the production of multiple molecular weights of HIV-1 envelope glycoprotein, rather than the usual GP-160, GP-120, and GP-41 electrophoretic bands. The APS also altered the viral envelope glycoproteins of New Castle and paramyxoviruses in culture which rendered the virions incapable of infecting susceptible target cell lines as well as animals.⁴

Alteration in critical viral envelope structures has therapeutic potential: There are 42 amino acids in the GP-120 segment of the HIV-1 viral envelope. Deletion of 12 amino acids in this region abolishes virion-binding capability to the target cell CD-4 receptor site and the substitution of only one amino acid reduces virion binding.⁵

GP-120 in the viral envelope is the critical glycoprotein that determines the ability of the virion to bind to the CD-4 receptors on susceptible cell membranes. This highly specific viral envelope receptor site binding is essential for infecting a host cell.⁶

The large APS molecules induced endoplasmic reticulum and Golgi dysynthesis of viral envelope glycoprotein and made the viral particle incapable of infecting susceptible target cells. A further immunomodulatory action of the APS is the increase in the monocytic/macrophagic (M/M) cell counts.' By contrast with Acemannan (900,000-1,500,000 Daltons), Polymannan Extract (PME) consists of an array of polysaccharide molecular species (50,000-10,000,000 Daltons) with the activity of the product largely related to the larger-sized molecular sizes. The discoverers of aloeride have suggested that the immunomodulatory action of Acemannan is actually due to trace contaminants with aloeride (2,000,000-10,000,000 Daltons).⁸

Aloe anthraquinones and AIDS: An Aloe vera leaf consists of a 15-18 layered outer rind with a protective xylan wax coating. Attached to the undersurface of the thick rind are the vascular bundles surrounded by the thick, gelly-like mucilage layer. The uppermost cells in the vascular bundle are the pericyclic cells which contain the yellow sap composed of a plethora of potent laxative-inducing anthraquinones, which served for several hundred years as laxative agents for both man and beast. These laxative agents have been largely replaced by “gentler” laxatives, but some veterinary use continues in some countries of the world.

Anthraquinones in Aloe barbadensis: Aloe-emodin (1,8-Dihydroxy-3-(hydroxymethyl-9,10-anthracenedione or 1,8-Dihydroxy-3-(hydroxymethyl)anthraquinone or 3-Hydroxymethylchrysazin) Aloe-Emodine-Anthranol, Aloinoside-A, Aloinoside-B, Aloin-A (10-Glucopyranosyl-1,S-dihydroxy-3-(hydroxymethyl)-9(10)-anthracene or 1,8-Dihydroxy-3-hydroxymethyl-10-(6-hydroxymethyl)-3,4,5-trihydroxy-2-pyranosyl) anthrone or 10-{1′,5′-anhydroglucosyl)-aloe-emodin-9-anthrone or Barbaloin), Aloin-B-Epimere of Aloin-A, Isobarbaloin, Aloinoslde-A, Aloinoside-B, Anthranols, Anthraquinone-glycoside, Chrysaminic Acid, Chrysophanic Acid, ChrysoDhanol, Chrysophanol-glycoside, 1,8-Dihydroxyanthracene, Emodin (1,3,8-Trihydroxy-6-methyl-9,10-anthracene-dione or 1,3,8-Trihydroxy-6-methylanthraquinone or 4,5,7-Trihydroxy-2-methylanthraquinone or Frangula emodin), Homonataloin, Hydroxymethylanthraquinone, Rhein (9,10-4,S-Dihydroxy-9,10-dioxo-2-anthracene-carboxylic acid or 1,5-Dihydroxyanthraquinone-3-carboxylic acid or 4,5-Dihydroxyanthraquanone-2-carboxylic acid or Chrysazin-3-carboxylic acid), Trihydroxymethylanthraquinone.

Anthraquinones may be selected from the list, used singly or in various combinations, or from compounds derived from the basic parent compound. In the AIDS therapy program, 50-75 mg of the selected anthraquinone(s) is administered orally to the patient on a daily basis, i.e., 25 mg b.i.d or t.i.d. These highly water-soluble moieties are readily absorbed, can enter the HIV-infected CD-4 cell and partially disrupt the viral capsular envelope, which completely prevents completion of the replication cycle. This modality obviates the limitation of various treatment modalities which are unable or enter to a very limited extent the virion-infected cell.⁹ This mechanism and the dysynthesis mechanism, of the large polysaccharides provides a deadly blow to the virions in the infected CD-4 cells.

Aloe vera, Natural Killer Cells and “M” Cell Functions: Aloe vera juice contains about 98% water and 2% total solids, which comprise of over 300 individual constituents, with the single largest component (10-12%) being glucomannan polysaccharides ranging from 50,000 Daltons to 100,000 Daltons.

When the aloe preparation (liquid or powder) is ingested, the polysaccharides do not undergo digestive breakdown by amylases, the sugar-digesting enzymes, because the glucose and mannose sugars are linked in the beta position. Thus, these large molecules can pass unchanged through the small intestine and the colon.

The small intestinal mucosa consists of approximately 97% fingerlike micro-villi, which significantly increase the surface area for the absorption of digested molecules. The remaining 3% of the lining cells are “M” (macromolecular) cells, which have a folded, undulating surface, which, through the process of endocytosis (cell-engulfment), permits large molecules and particles to be taken up. When the large polysaccharide molecules exit from the base of the “M” cell, they immediately come into contact with mononuclear white blood cells, (macrophages/monocytes) components of the immune system.

The polysaccharides attach to the mannose receptors on the surface of the white cell, which causes the elaboration of a family of immune system factors including Tumor Necrosis Factor-alpha (TNF-α), Interleukin-I_{β} (IL-β), lnterleukin-2 (IL-2), Interleukin-6 (IL-6), and gamma interferon (IFN-γ).¹⁰

Interleukin-2 (IL-2) stimulates the growth and potentiates the cytolytic action of Natural Killer (NK) cells through the generation of lymphokine-activated killer (LAK) cells, which induce differentiation and proliferation processes. Gamma interferon (INF-γ) is a powerful activator of NK cells.

In AIDS patients the number of “M” cells may increase significantly (as also occurs with other disease processes involving the gastrointestinal tract) even up to 15-18% compared with the normal 3%. (a) Opportunistic organisms residing in the bowel lumen may gain entrance to the blood stream through the “M” cell endocytotic uptake. (b) The large molecular polysaccharides in the freeze-dried aloe powder, administered in capsules, passes undigested through the small bowel (owing to the beta-linkage of the substituent sugars), and attach to the mannose-binding sites on the “M” cells. They are taken in to the “M” cells by endocytosis and exit the “M” cell base where they encounter monocyte/macrophage (M/M) cells of the immune system where they initiate the secretion of the suite of cytocommunicators (TNF-α, IL-1β, IL-2, IL-6, INF-γ) similar to the action of injected PME administered intravenously. (c) Unabsorbed large polysaccharide molecules occupy the endocytic attachment sites, and owing to their large size remain in place for several hours thereby obviating the uptake of opportunistic agents.

Natural Killer Cells (NK): (i) NK. cells are lymphoid cells of the natural immune system that express cytotoxicity against various nucleated cells including tumor cells and virus-infected cells, (ii) NK cells are believed to represent a significant part of the natural immune defense against spontaneously-developing neoplastic cells and against infection by viruses, (iii) NK. cells do not require programming by prior contact with antigens and are not restricted by the major histocompatability complex (MHC) antigens, (iv) Although NK cells are not phagocytic, they are capable of attacking and destroying malignant tumor cells, (v) Over 150 different types of white cells have been identified and, of these, NK cells are one of the most common, representing up to 15% of the total white blood cells. Unlike other white cells, they are able to work more or less independently (less programmed), not requiring special instructions from the immune system in order to recognize or attack a foreign cell. Thus, they often are considered to be body's first line of defense against cancer and viral-infected cells, (vi) Circulating through the body by way of the blood and lymph, the majority of NK cells present in the body are in a resting but ready state: (a) NK cells are activated by immunoregulatory proteins called cytokines Once activated, the NK cells become quite rapacious in their search-and-destroy activities, (b) Upon encountering a tumor cell, the activated NK cell attaches to the membrane of the cancer or viral cell and injects cytoplasmic granules that quickly dissolve (lyse) the target cell., (c) In less than five minutes the cancer cell is dead and the NK cell moves on to its next victim, (d) Although much smaller in comparison to tumor or viral cells, a single NK cell can often bind to two or more cancer cells at once, (e) A single NK cell can destroy up to 27 cancer cells before it dies, (vii) The absolute number of NK cells present in the blood gives little indication of efficacy of immune function: (a) Instead, it is NK cell activity—the avidity with which they recognize and bind to tumor or viral cells that is important, (b) Most immunomodulatory substances can empower the NK cells to accentuate their degree of activity, (c) In healthy immunocompetent individuals, when NK cell activity is examined at an effector target ratio of 100:1, NK cell activity ranges from 60-75%, (d) In the cancer patient, NK activity typically ranges from 0 to 30%; the reasons for this significant decline are not well understood and remain elusive.

Summary of Therapeutic Mechanisms in AIDS

I. Polymannan Extract (PME): (a) Inducement of dysynthesis of GP-120 moiety of the virion envelope which abrogates the capacity of the virion to bind to the CD-4 receptor, (b) Increase in the number of T4 helper cells, (c) Upon intravenous administration, the large glucomannan polysaccharides attach to mannose receptors on the monocyte/macrophage cells which induces the elaboration and release of a suite of cytocommunicators including TNF-α, IL-1β, IL-2, IL-6, and INF-γ. The IL-2 stimulates the growth and potentiates the cytolytic action of the Natural Killer (NK) cells via the generation of lymphokine-activated killer cells (LAF) which induce differentiation and proliferative processes. INF-γ is a powerful activator of NK cells.

II. Raidox: (a) The anthraquinone mixture enters the infected T4-helper cells and partially disrupts the viral envelope, which abrogates the replication cycle. This action is in addition to the dysynthesis of the GP-120 glycoprotein.

III. Aloe vera freeze dried powder: (a) The large polysaccharides (PS) in aloe liquids, gels, and powders consist of simple hexoses (mannose, glucose, arabinose, and galactose) linked together in the beta position which protects these molecules from being amenable to digestion by amylases leaving these long-chain molecules intact, (b) Oral administration of the powder (contains over 300 constituents with the long polysaccharides being the most prominent (10-12% of the total solids)) permits the PS to bind to the small intestinal “M” cells and induce the release of the cytocommunicator suite from the M/M located at the base of the “M” cells, (c) The large PS molecules may remain bound to the “M” cell mucosal surface for several hours, thereby blocking the uptake by the “M” cells of opportunistic entities responsible for the untimely deaths of AIDS patients.

Products

I. Polymannan Extract (PME): (a) Available as a sterile solution for injection (i.m. and i.v.), (b) Concentration-10 mg/ml, (c) Available in 10 ml multidose vials, and (d) Preservative-0.9% Benzyl alcohol, if desired

II. Raldox-R (Anthraquinones): (a) Available as a selected mixture of anthraquinones, (b) Available in vegcaps each containing 25 mg, (c) Available in bottles of 60 capsules

III. Freeze-Dried Aloe vera Powder: (a) Produced under U.S. Pat. No. 6,083,508, (b) Contains no preservatives, (c) Available in vegcaps each containing total processed lyophilized aloe vera 400 mg/capsule.

IV. Nutritional Supplements Many AIDS patients especially those who experience chronic diarrhea and digestive upsets associated with weight loss, develop malnutrition. Two products are available:

(a) GLE/EPA Capsule Ingredients

| | |
|---|---|
| Linoleic Acid (LA) | 280 mg |
| Gamma Linolenic Acid (GLA) | 80 mg |
| Eicosapentaenoic Acid (EPA) | 45 mg |
| Doscosapentaenoic Acid (DPA) | 9 mg |
| Docosahexaenoic Acid (DHA) | 30 mg |
| d'alpha tocopherol | 15 mg |

(b) Powder Ingredients

Nutritional Information per serving

| Serving Size | 48 | grams | Fat | 1 | gm |
|---|---|---|---|---|---|
| Calories | 160 | | *Cholesterol | 0 | gm |
| Protein | 13 | gms | Sodium | 120 | mg |
| Carbohydrates | 26 | gms | Potassium | 660 | mg |

Percentages of U.S. Recommended Daily Allowances (U.S. RDA) per serving:

| Protein | 30% | Vitamin E | 50% | Copper | 50% | |
|---|---|---|---|---|---|---|
| Vitamin A | 70% | Vitamin B₆ | 50% | Biotin | 50% | |
| Vitamin C | 50% | Folic Acid | 50% | Pantothenic Acid | 50% | |
| Thiamine | 50% | Vitamin B₁₂ | 50% | **Chromium | 23 | mcg |
| Riboflavin | 60% | Phosphorus | 20% | **Selenium | 23 | mcg |
| Niacin | 50% | Iodine | 50% | **Manganese | 2 | mg |
| Calcium | 25% | Magnesium | 50% | **Fiber | 3 | gms |
| Iron | 50% | Zinc | 50% | **Octacosanol | 2000 | mcg |
| Vitamin D | 50% | | | | | |

Amino Acids per 48 gram serving

| | | | | | | | | |
|---|---|---|---|---|---|---|---|---|
| Alanine | 375 | mg | Histidine | 320 | mg | Proline | 1425 | mg |
| Arginine | 450 | mg | Isoleucine | 655 | mg | Serine | 700 | mg |
| Aspartic acid | 850 | mg | ***Leucine | 1115 | mg | ***Threonine | 510 | mg |
| Cystine | 65 | mg | ***Lysine | 920 | mg | ***Tryptophan | 145 | mg |
| Glutamic acid | 2640 | mg | ***Methionine | 310 | mg | Tyrosine | 630 | mg |
| Glycine | 250 | mg | ***Phenylalanine | 600 | mg | ***Valine | 755 | mg |

Ingredients:
Fructose, Nonfat Milk Solids, Calcium Sodium Caseinate, Natural and Artificial flavors, Cellulose, Corn Bran, Potassium Chloride, Lecithin, Maltodextrin, Carrageenan, Wheat Germ Oil, (source of Octacosanol), Magnesium Oxide, Beta Carotene, Selenium, Ascorbic Acid, Ferrous Fumarate, d'Alpha Tocopherol Acetate, Chromium, Niacin, Aloe, Apple Pectin, Zinc Oxide, Manganese Sulfate, Vitamin A Palmitate, d-Calcium Pantothenate, Copper Sulfate, Pyridoxine Hydrochloride, Riboflavin, Thiamine Hydrochloride, Cobalamin Concentrate, Vitamin D₂, Folic Acid, Biotin and Potassium Chloride.
*Information on cholesterol content is provided for individuals who, on the advice of a physician are modifying their dietary intake of cholesterol.
**No RDA established for these nutrients.
***Essential Amino Acids

Instructions for use of Polymannan Extract

The diagnosis must be confirmed by standard laboratory and other pertinent diagnostic procedures, e.g. CD4 cell count and circulating viral load in AIDS/HIV, PSA in prostate cancer, pathological assay of biopsy specimens, tumor markers, etc. The PME comprises long-chain partially acetylated glucomannan polysaccharides of botanical origin (Aloe barbadensis) having a molecular range of about 100,000 Daltons to 10,000,000 Daltons. This extract possesses very potent immunomodulatory actions. Upon intravenous injection, the PME molecules bind to mannose receptors on the surface membranes of monocytes/macrophages, which results in the secretory elaboration of an array of cytocommunicators, e.g., cytokines, interleukins, interferons, prostaglandins, growth factors, etc., the profile of which varies depending upon the disease entity present. These then, bring about salubriously therapeutic responses in malignant neoplasms, gastrointestinal diseases (e.g., hepatitis C, Chronic ulcerative Colitis, Crohn's Disease, etc.), viral diseases (e.g., Epstein-Barr, Lyme's Disease, etc.), as well as increasing beta-lymphocytes and antibodies, and increased levels of natural killer cells.

Additional mannose binding sites are found on the circulating protein—Mannose Binding Protein (MBP) which functions as a donor to nascent monocyte/macrophage cellular elements entering the circulatory system, owing to a greater affinity for PME of the mannose receptors on these cells. Studies have shown that, once PME has been bound to the monocyte/macrophage cellular element, bonding remains in place for about 48 hours, which forms the basis for the thrice weekly intravenous administration treatment schedule.

The PME product is available as a sterile solution for injection in a multi-dose 10 mL vial with a concentration of 10 mg/mL. Owing to the exceptional potency of the product, the beginning dosage is 1 mg (0.1 mL). As the product contains no preservatives, exceptionally stringent aseptic conditions must be employed in the administration of PME.

Physiological Reaction: Upon intravenous administration, if sufficient mannose-binding sites are occupied and the resulting cytocommunicator release is adequate, several proinflammatory moieties, e.g., Tumor Necrosis Factors-alpha and Interleukin-1β are released which bring about an acute phase response (APR) manifested by changes in the hypothalamic temperature-regulating centers, resulting in sensations of chilliness, coldness, shivering, shakes, and rigors associated with the attempt to satisfy the cytocommunicator- induced resetting of the “thermostat.” This is an expected and desirable physiological reaction, not a side-effect, as this indicates that the immunomodulatory activities of the monocyte/macrophage cellular elements have been stimulated. The physiological reaction may begin as soon as 30-minutes after injection or take as long as 3-4 hours. Once begun, the entire set of symptoms is usually abrogated within 1.5 to 2 hours following administration.

In some patients the reaction may produce excessive anxiety. In these situations, the PME may be added to a D-5-W or Normal Saline intravenous infusion, which permits a slower administration with a lessening of acuity of the physiological response.

If the reactions are still bothersome, the patient may be premedicated an hour before administration using an NSAID, (e.g., 400-800 mg Ibuprofen, or similar), which obtunds the symptoms while in no way impairing the resulting immunomodulatory actions induced by the injection.

Factors Influencing the Physiological Response: (i) White blood cell count, (ii) WBC differential respecting the monocyte/macrophage cell count, (iii) Number of mannose receptors per monocyte/macrophage, (iv) Proper stereochemical configuration of the mannose receptor to enable attachment of the polysaccharide molecules. In some patients who have previous received chemotherapy, mannose receptor function may be seriously compromised.

Treatment Schedule: For the initial treatment, 1 mg (0.1 mL) of PME is given one Day 1. On each successive day, the dosage is increased by 0.1 mL (1 milligram), i.e., 0.5 mL (5 milligrams) on Day 5, or until the physiological response is manifest. This procedure is to assure optimal mannose-receptor binding on monocytes/macrophages and the Mannose Binding Protein circulating carrier, and to determine the dosage necessary to stimulate an adequate response from the immune system.

Thereafter, treatments are given three times a week increasing the dosage by 1 mg (0.1 mL) of the PME as may be required to maintain a physiological reaction, at least, of some perceptible degree.

Most patients eventually stabilize on a treatment dosage of 10 to 15 mg (1.0-1.5 mL) tri weekly. Each patient must be titrated, as each person shows a variability in response to the PME related to variations in WBCs, differential, number of mannose receptors, etc.

Side-effects: Side-effects, including nausea (very occasional vomiting and/or a slight degree of hypotension), occur only with excessive dosages, and with careful management can be entirely avoided. Mild hypotension occurs after the administration of excessively large doses (greater than recommended) which was seen in the Phase I Safety Study. Some patients have experienced some joint pain with large doses. Mention has been made previously of administering PME as an infusion of D-5-W or Normal Saline, and/or pre-administration oral ingestion of NSAID medication.

Clinical and Laboratory Evaluations: As each patient is quite different in the response to PME injections, each patient must be titrated both with respect to the treatment frequency and dosage, and the frequency of clinical and laboratory investigations to assess the progress of the patient and the character of the response to PME administration. Other treatment modalities, e.g., nutritional supplements, low-dose chemotherapeutics, psychological conditioning, etc. may often be employed with additional improvement on the part of the patient.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

U.S. Pat. No. 7,169,414: Antimicrobial Agents Isolated from Aloe Vera.

WIPO Patent No. WO/1993/008810: Uses of Aloe Products, e.g. Acemannan, in the Treatment of Diseases Requiring Intervention of the Immune System for Cure.

¹ Pulse T L, Uhlig E: A Significant Improvement in Clinical Pilot Study Utilizing Nutritional Supplements, Essential Fatty Acids and Stabilized Aloe Vera Juice in 29 HIVSeropositive, ARC and AIDS Patients. Journal of Advancement in Medicine 3(4), 1990.

² Leung M Y, Liu C, Zhu L F, Hui V Z, Fung KP: Chemical and biological characterization of a polysaccharide biological response modifier from Aloe vera. Glycobiology 14(6):501-510, 2004;

³ Liu C, Leung M Y, Koon J C, Zhu L F, Hui V Z, Yu B, Fung KP: Macrophage activation by polysaccharide biological response modifiers isolated from Aloe vera 1. var. chinensis (Haw.) Berg. International Immunopharmacology 6 {11):634-641, 2006)

⁴ Mitchell W M: In vitro evaluation of Acemannan as an anti-HIV-1 agent. Carrington Laboratories Research Report 40001-December, 1987.

⁵ Laskey L A, Nakamura G, Smith D H et al: Delineation of a region of the human immunodeficiency virus Type 1 GP120 glycoprotein critical for interaction with the CD-4 receptor. Cell SO:975-985, 1987.

⁶ Chinna A D: Evaluation of the antiviral, adjuvant, and immunomodulatory effect of b-(1,4)-lineal polymannose (Acemannan). Texas A&M University, College Station, Tex. Diss. Abstr., 1990.

⁷ McDaniel H R et al: An increase in circulating monocyte/macrophage (M/M) is induced by Acemannan (ACE-m) in HIV-1 patients. Amer J Path 94:516-517,1990.

⁸ Pugh N, Ross S A, ElSohly M A, Pasco D S: Characterization of aloeride, a new high-molecular weight polysaccharide from Aloe vera with potent immuno-stimulating activity. J Agric Food Chem 49(2):1032-1034, 2001.

⁹ Sydiskis R J, Owen D G, Lohr J L et al: Interaction of enveloped virus by anthraquinones extracted from plants. Antimicrobial Agents and Chemotherapeutics 3S:2463-2464, 1991.

¹⁰ M Y Leung, C Liu, L F Zhu, Y Z Hui, K P Fung Chemical and biological characterization of a polysaccharide biological response modifier from Aloe vera L. Glycobiology 14(6):501-510, 2004.

1. A pharmaceutical composition for treating, amelioration of symptoms, or both of Acquired Immune Deficiency Syndrome (AIDS) or a HIV infection in a subject comprising:
a sterile injectable polymannan extract formulation comprising one or more aloe polysaccharides, wherein the aloe polysaccharides comprise one or more small chain, medium chain, large chain, very-large chain polysaccharides, or any combinations thereof;
a formulation comprising one or more aloe anthraquinones, diacetyl anthraquinone derivatives, and combinations and modifications thereof;
a formulation comprising a freeze dried aloe powder, an aloe juice, an aloe gel, or a combination wherein the powder is administered as is or after reconstitution in a suitable liquid; and
a formulation comprising one or more optional nutritional supplements selected from the group consisting of fatty acids, proteins, minerals and metals, vitamins, salts, amino acids, and other pharmaceutically acceptable excipients.

2. The composition of claim 1, wherein the polymannan extract is administered intravenously, intramuscularly, subcutaneously, intraperitoneally, or by any other suitable parenteral route.

3. The composition of claim 1, wherein the polymannan extract is administered intravenously.

4. The composition of claim 1, wherein the polymannan extract is administered at a dose of 10-15 mg three times a week.

5. The composition of claim 1, wherein the formulations other than the polymannan extract are administered orally.

6. The composition of claim 1, wherein the one or more aloe polysaccharides in the polymannan extract have molecular weights ranging from 50,000-10,000,000 Daltons.

7. The composition of claim 1, wherein the one or more aloe anthraquinones are selected from the group consisting of Aloe-emodin, 1,8-Dihydroxy-3-(hydroxymethyl-9,10-anthracenedione, 1,8-Dihydroxy-3-(hydroxymethyl)anthraquinone, 3-Hydroxymethylchrysazin, Aloe-Emodine-Anthranol, Aloinoside-A, Aloinoside-B, Aloin-A, 10-Glucopyranosyl-1,S-dihydroxy-3-(hydroxymethyl)-9(10)-anthracene,1,8-Dihydroxy-3-hydroxymethyl-10-(6-hydroxymethyl)-3,4,5-trihydroxy-2-pyranosyl, anthrone, 10-{1′,5′-anhydroglucosyl)-aloe-emodin-9-anthrone, Barbaloin, Aloin-B Epimere of Aloin-A, Isobarbaloin, Aloinoslde-A, Aloinoside-B, Anthranols, Anthraquinone-glycocide, Chrysaminic Acid, Chrysophanic Acid, ChrysoDhanol, Chrysophanol-glycoside, 1,8-Dihydroxyanthracene, Emodin, 1,3,8-Trihydroxy-6-methyl-9,10-anthracene-dione, 1,3,8-Trihydroxy-6-methylanthraquinone, 4,5,7-Trihydroxy-2-methylanthraquinone, Frangula emodin, Homonataloin, Hydroxymethylanthraquinone, Rhein, 9,10-4,5-Dihydroxy-9,10-dioxo-2-anthracene-carboxylic acid, 1,5-Dihydroxyanthraquinone-3-carboxylic acid, 4,5-Dihydroxyanthraquanone-2-carboxylic acid, Chrysazin-3-carboxylic acid, Trihydroxymethylanthraquinone, and modifications and derivatives thereof.

8. The composition of claim 1, wherein the aloe anthraquinones are administered at a dose ranging from 50-75 mg/day.

9. The composition of claim 1, wherein the freeze dried aloe powder, the aloe juice, the aloe gel or the combination is administered at a dose of 400 mg one or more times a day.

10. The composition of claim 1, wherein the aloe juice comprises of about 2% total solids.

11. The composition of claim 1, wherein the aloe juice comprises glucomannan polysaccharides.

12. The composition of claim 11, wherein the glucomannan polysaccharides have molecular weights ranging from 50,000-10,000,000 Daltons.

13. The composition of claim 1, wherein the one or more fatty acids are selected from the group consisting of Linoleic Acid (LA), Gamma-linolenic Acid (GLA), Eicosapentaneoic Acid (EPA), Docosapentaneoic Acid (DPA), Docosahexaenoic CAid (DHA), and D-alpha-tocopherol.

14. The composition of claim 1, wherein the composition releases one or more cytocommunicators selected from the group consisting of TNF-α, IL-1β, IL-2, IL-6, and INF-γ, wherein the release stimulates the growth and cytolytic action of one or more Natural Killer (NK) cells in the subject.

15. A method of treating, ameliorating symptoms or both of Acquired Immune Deficiency Syndrome (AIDS) or a HIV infection in a subject comprising the steps of:
identifying the subject in need of the treatment or amelioration of the symptoms against the AIDS or the HIV infection; and
administering a therapeutically effective amount of a pharmaceutical composition sufficient to treat or ameliorate the symptoms of the AIDS or the HIV infection, wherein the pharmaceutical composition comprises:
a sterile injectable polymannan extract formulation comprising one or more aloe polysaccharides, wherein the aloe polysaccharides comprise one or more small chain, medium chain, large chain, very-large chain polysaccharides, or any combinations thereof;
a formulation comprising one or more aloe anthraquinones, diacetyl anthraquinone derivatives, and combinations and modifications thereof;
a formulation comprising a freeze dried aloe powder, an aloe juice, an aloe gel or a combination wherein the powder is administered as is or after reconstitution in a suitable liquid; and
a formulation comprising one or more optional nutritional supplements selected from the group consisting of fatty acids, proteins, minerals and metals, vitamins, salts, amino acids, and other pharmaceutically acceptable excipients.

16. The method of claim 15, wherein the polymannan extract is administered intravenously.

17. The method of claim 15, wherein the polymannan extract is administered at a dose of 10-15 mg three times a week.

18. The method of claim 15, wherein the formulations other than the polymannan extract are administered orally.

19. The method of claim 15, wherein the one or more aloe anthraquinones are selected from the group consisting of Aloe-emodin, 1,8-Dihydroxy-3-(hydroxymethyl-9,10-anthracenedione, 1,8-Dihydroxy-3-(hydroxymethyl)anthraquinone, 3-Hydroxymethylchrysazin, Aloe-Emodine-Anthranol, Aloinoside-A, Aloinoside-B, Aloin-A, 10-Glucopyranosyl-1,S-dihydroxy-3-(hydroxymethyl)-9(10)-anthracene,1,8-Dihydroxy-3-hydroxymethyl-10-(6-hydroxymethyl)-3,4,5-trihydroxy-2-pyranosyl, anthrone, 10-{1,5′-anhydroglucosyl)-aloe-emodin-9-anthrone, Barbaloin, Aloin-B Epimere of Aloin-A, Isobarbaloin, Aloinoslde-A, Aloinoside-B, Anthranols, Anthraquinone-glycocide, Chrysaminic Acid, Chrysophanic Acid, ChrysoDhanol, Chrysophanol-glycoside, 1,8-Dihydroxyanthracene, Emodin, 1,3,8-Trihydroxy-6-methyl-9,10-anthracene-dione, 1,3,8-Trihydroxy-6-methylanthraquinone, 4,5,7-Trihydroxy-2-methylanthraquinone, Frangula emodin, Homonataloin, Hydroxymethylanthraquinone, Rhein, 9,10-4,5-Dihydroxy-9,10-dioxo-2-anthracene-carboxylic acid, 1,5-Dihydroxyanthraquinone-3-carboxylic acid, 4,5-Dihydroxyanthraquanone-2-carboxylic acid, Chrysazin-3-carboxylic acid, Trihydroxymethylanthraquinone, and modifications and derivatives thereof.

20. The method of claim 15, wherein the aloe anthraquinones are administered at a dose ranging from 50-75 mg/day.

21. The method of claim 15, wherein the aloe juice comprises glucomannan polysaccharides.

22. The method of claim 15, wherein the freeze dried aloe powder, the aloe juice, the aloe gel or the combination is administered at a dose of 400 mg one or more times a day.

23. The method of claim 15, wherein the one or more fatty acids are selected from the group consisting of Linoleic Acid (LA), Gamma-linolenic Acid (GLA), Eicosapentaneoic Acid (EPA), Docosapentaneoic Acid (DPA), Docosahexaenoic CAid (DHA), and D-alpha-tocopherol.

24. The method of claim 15, wherein the composition releases one or more cytocommunicators selected from the group consisting of TNF-α, IL-1β, IL-2, IL-6, and INF-γ, wherein the release stimulates the growth and cytolytic action of one or more Natural Killer (NK) cells in the subject.

25. A pharmaceutical composition for treating, amelioration of symptoms or both of Acquired Immune Deficiency Syndrome (AIDS) or a HIV infection in a subject comprising:
a sterile injectable polymannan extract formulation comprising one or more aloe polysaccharides, wherein the aloe polysaccharides comprise one or more small chain, medium chain, large chain, very-large chain polysaccharides, or any combinations thereof;
a formulation comprising one or more aloe anthraquinones, diacetyl anthraquinone derivatives, and combinations and modifications thereof;
a formulation comprising a freeze dried aloe powder, an aloe juice, an aloe gel or a combination wherein the powder is administered as is or after reconstitution in a suitable liquid; and
a formulation of a nutritional supplement comprising:

| | |
|---|---|
| Linoleic Acid (LA) | 280 mg |
| Gamma Linolenic Acid (GLA) | 80 mg |
| Eicosapentaenoic Acid (EPA) | 45 mg |
| Doscosapentaenoic Acid (DPA) | 9 mg |
| Docosahexaenoic Acid (DHA) | 30 mg |
| d'alpha tocopherol | 15 mg |

26. The composition of claim 25, wherein the polymannan extract is administered intravenously.

27. The composition of claim 25, wherein the formulations other than the polymannan extract are administered orally.

28. The composition of claim 25, wherein the one or more aloe anthraquinones are selected from the group consisting of Aloe-emodin, 1,8-Dihydroxy-3-(hydroxymethyl-9,10-anthracenedione, 1,8-Dihydroxy-3-(hydroxymethyl)anthraquinone, 3-Hydroxymethylchrysazin, Aloe-Emodine-Anthranol, Aloinoside-A, Aloinoside-B, Aloin-A, 10-Glucopyranosyl-1,S-dihydroxy-3-(hydroxymethyl)-9(10)-anthracene, 1,8-Dihydroxy-3-hydroxymethyl-10-(6-hydroxymethyl)-3,4,5-trihydroxy-2-pyranosyl, anthrone, 10-{1′,5′-anhydroglucosyl)-aloe-emodin-9-anthrone, Barbaloin, Aloin-B Epimere of Aloin-A, Isobarbaloin, Aloinoslde-A, Aloinoside-B, Anthranols, Anthraquinone-glycocide, Chrysaminic Acid, Chrysophanic Acid, ChrysoDhanol, Chrysophanol-glycoside, 1,8-Dihydroxyanthracene, Emodin, 1,3,8-Trihydroxy-6-methyl-9,10-anthracene-dione, 1,3,8-Trihydroxy-6-methylanthraquinone, 4,5,7-Trihydroxy-2-methylanthraquinone, Frangula emodin, Homonataloin, Hydroxymethylanthraquinone, Rhein, 9,10-4,5-Dihydroxy-9,10-dioxo-2-anthracene-carboxylic acid, 1,5-Dihydroxyanthraquinone-3-carboxylic acid, 4,5-Dihydroxyanthraquanone-2-carboxylic acid, Chrysazin-3-carboxylic acid, Trihydroxymethylanthraquinone, and modifications and derivatives thereof.

29. The composition of claim 25, wherein the aloe juice comprises glucomannan polysaccharides.

30. A method of treating, ameliorating symptoms or both of an Acquired Immune Deficiency Syndrome (AIDS) or a HIV infection in a subject comprising the steps of:
identifying the subject in need of the treatment or amelioration of the symptoms against the AIDS or the HIV infection; and
administering a therapeutically effective amount of a pharmaceutical composition sufficient to treat or ameliorate the symptoms of the AIDS or the HIV infection, wherein the pharmaceutical composition comprises:
a sterile injectable polymannan extract formulation comprising one or more aloe polysaccharides, wherein the aloe polysaccharides comprise one or more small chain, medium chain, large chain, very-large chain polysaccharides, or any combinations thereof;
a formulation comprising one or more aloe anthraquinones, diacetyl anthraquinone derivatives, and combinations and modifications thereof;
a formulation comprising a freeze dried aloe powder, an aloe juice, an aloe gel or a combination wherein the powder is administered as is or after reconstitution in a suitable liquid; and
a formulation of a nutritional supplement comprising:

| | |
|---|---|
| Linoleic Acid (LA) | 280 mg |
| Gamma Linolenic Acid (GLA) | 80 mg |
| Eicosapentaenoic Acid (EPA) | 45 mg |
| Doscosapentaenoic Acid (DPA) | 9 mg |
| Docosahexaenoic Acid (DHA) | 30 mg |
| d'alpha tocopherol | 15 mg |

31. The method of claim 0, wherein the polymannan extract is administered intravenously at a dose of 10-15 mg three times a week.

32. The method of claim 0, wherein the formulations other than the polymannan extract are administered orally.

33. The method of claim 0, wherein the aloe anthraquinones are administered at a dose ranging from 50-75 mg/day.

34. The method of claim 0, wherein the freeze dried aloe powder, the aloe juice, the aloe gel or the combination is administered at a dose of 400 mg one or more times a day.

35. The method of claim 0, wherein the composition releases one or more cytocommunicators selected from the group consisting of TNF-α, IL-13, IL-2, IL-6, and INF-γ, wherein the release stimulates the growth and cytolytic action of one or more Natural Killer (NK) cells in the subject.

36. A pharmaceutical composition for treating, amelioration of symptoms or both of Acquired Immune Deficiency Syndrome (AIDS) or a HIV infection in a subject comprising:
a sterile injectable polymannan extract formulation comprising one or more aloe polysaccharides, wherein the aloe polysaccharides comprise one or more small chain, medium chain, large chain, very-large chain polysaccharides, or any combinations thereof;
a formulation comprising one or more aloe anthraquinones, diacetyl anthraquinone derivatives, and combinations and modifications thereof;
a formulation comprising a freeze dried aloe powder, an aloe juice, an aloe gel or a combination wherein the powder is administered as is or after reconstitution in a suitable liquid; and
a formulation of a nutritional supplement comprising:
Nutritional Information per serving

| Serving Size | 48 | grams | Fat | 1 | gm |
|---|---|---|---|---|---|
| Calories | 160 | | *Cholesterol | 0 | gm |
| Protein | 13 | gms | Sodium | 120 | mg |
| Carbohydrates | 26 | gms | Potassium | 660 | mg |

Percentages of U.S. Recommended Daily Allowances (U.S. RDA) per serving:

| Protein | 30% | Vitamin E | 50% | Copper | 50% | |
|---|---|---|---|---|---|---|
| Vitamin A | 70% | Vitamin B₆ | 50% | Biotin | 50% | |
| Vitamin C | 50% | Folic Acid | 50% | Pantothenic Acid | 50% | |
| Thiamine | 50% | Vitamin B₁₂ | 50% | **Chromium | 23 | mcg |
| Riboflavin | 60% | Phosphorus | 20% | **Selenium | 23 | mcg |
| Niacin | 50% | Iodine | 50% | **Manganese | 2 | mg |
| Calcium | 25% | Magnesium | 50% | **Fiber | 3 | gms |
| Iron | 50% | Zinc | 50% | **Octacosanol | 2000 | mcg |
| Vitamin D | 50% | | | | | |

Amino Acids per 48 gram serving

| | | | | | | | | |
|---|---|---|---|---|---|---|---|---|
| Alanine | 375 | mg | Histidine | 320 | mg | Proline | 1425 | mg |
| Arginine | 450 | mg | Isoleucine | 655 | mg | Serine | 700 | mg |
| Aspartic acid | 850 | mg | ***Leucine | 1115 | mg | ***Threonine | 510 | mg |
| Cystine | 65 | mg | ***Lysine | 920 | mg | ***Tryptophan | 145 | mg |
| Glutamic acid | 2640 | mg | ***Methionine | 310 | mg | Tyrosine | 630 | mg |
| Glycine | 250 | mg | ***Phenylalanine | 600 | mg | ***Valine | 755 | mg |

Ingredients:
Fructose, Nonfat Milk Solids, Calcium Sodium Caseinate, Natural and Artificial flavors, Cellulose, Corn Bran, Potassium Chloride, Lecithin, Maltodextrin, Carrageenan, Wheat Germ Oil, (source of Octacosanol), Magnesium Oxide, Beta Carotene, Selenium, Ascorbic Acid, Ferrous Fumarate, d'Alpha Tocopherol Acetate, Chromium, Niacin, Aloe, Apple Pectin, Zinc Oxide, Manganese Sulfate, Vitamin A Palmitate, d-Calcium Pantothenate, Copper Sulfate, Pyridoxine Hydrochloride, Riboflavin, Thiamine Hydrochloride, Cobalamin Concentrate, Vitamin D₂, Folic Acid, Biotin and Potassium Chloride.

37. The composition of claim 36, wherein the polymannan extract is administered intravenously.

38. The composition of claim 36, wherein the formulations other than the polymannan extract are administered orally.

39. The composition of claim 36, wherein the one or more aloe anthraquinones are selected from the group consisting of Aloe-emodin, 1,8-Dihydroxy-3-(hydroxymethyl-9,10-anthracenedione, 1,8-Dihydroxy-3-(hydroxymethyl)anthraquinone, 3-Hydroxymethylchrysazin, Aloe-Emodine-Anthranol, Aloinoside-A, Aloinoside-B, Aloin-A, 10-Glucopyranosyl-1,S-dihydroxy-3-(hydroxymethyl)-9(10)-anthracene, 1,8-Dihydroxy-3-hydroxymethyl-10-(6-hydroxymethyl)-3,4,5-trihydroxy-2-pyranosyl, anthrone, 10-{1′,5′-anhydroglucosyl)-aloe-emodin-9-anthrone, Barbaloin, Aloin-B Epimere of Aloin-A, Isobarbaloin, Aloinoslde-A, Aloinoside-B, Anthranols, Anthraquinone-glycocide, Chrysaminic Acid, Chrysophanic Acid, ChrysoDhanol, Chrysophanol-glycoside, 1,8-Dihydroxyanthracene, Emodin, 1,3,8-Trihydroxy-6-methyl-9,10-anthracene-dione, 1,3,8-Trihydroxy-6-methylanthraquinone, 4,5,7-Trihydroxy-2-methylanthraquinone, Frangula emodin, Homonataloin, Hydroxymethylanthraquinone, Rhein, 9,10-4,5-Dihydroxy-9,10-dioxo-2-anthracene-carboxylic acid, 1,5-Dihydroxyanthraquinone-3-carboxylic acid, 4,5-Dihydroxyanthraquanone-2-carboxylic acid, Chrysazin-3-carboxylic acid, Trihydroxymethylanthraquinone, and modifications and derivatives thereof.

40. The composition of claim 36, wherein the aloe juice comprises glucomannan polysaccharides.

41. A method of treating, ameliorating symptoms or both of Acquired Immune Deficiency Syndrome (AIDS) or a HIV infection in a subject comprising the steps of:
identifying the subject in need of the treatment or amelioration of the symptoms against the AIDS or the HIV infection; and
administering a therapeutically effective amount of a pharmaceutical composition sufficient to treat or ameliorate the symptoms of the AIDS or the HIV infection, wherein the pharmaceutical composition comprises:
a sterile injectable polymannan extract formulation comprising one or more aloe polysaccharides, wherein the aloe polysaccharides comprise one or more small chain, medium chain, large chain, very-large chain polysaccharides, or any combinations thereof;
a formulation comprising one or more aloe anthraquinones, diacetyl anthraquinone derivatives, and combinations and modifications thereof;
a formulation comprising a freeze dried aloe powder, an aloe juice, an aloe gel or a combination wherein the powder is administered as is or after reconstitution in a suitable liquid; and
a formulation of a nutritional supplement comprising:
Nutritional Information per serving

| Serving Size | 48 | grams | Fat | 1 | gm |
|---|---|---|---|---|---|
| Calories | 160 | | *Cholesterol | 0 | gm |
| Protein | 13 | gms | Sodium | 120 | mg |
| Carbohydrates | 26 | gms | Potassium | 660 | mg |

Percentages of U.S. Recommended Daily Allowances (U.S. RDA) per serving:

| Protein | 30% | Vitamin E | 50% | Copper | 50% | |
|---|---|---|---|---|---|---|
| Vitamin A | 70% | Vitamin B₆ | 50% | Biotin | 50% | |
| Vitamin C | 50% | Folic Acid | 50% | Pantothenic Acid | 50% | |
| Thiamine | 50% | Vitamin B₁₂ | 50% | **Chromium | 23 | mcg |
| Riboflavin | 60% | Phosphorus | 20% | **Selenium | 23 | mcg |
| Niacin | 50% | Iodine | 50% | **Manganese | 2 | mg |
| Calcium | 25% | Magnesium | 50% | **Fiber | 3 | gms |
| Iron | 50% | Zinc | 50% | **Octacosanol | 2000 | mcg |
| Vitamin D | 50% | | | | | |

Amino Acids per 48 gram serving

| | | | | | | | | |
|---|---|---|---|---|---|---|---|---|
| Alanine | 375 | mg | Histidine | 320 | mg | Proline | 1425 | mg |
| Arginine | 450 | mg | Isoleucine | 655 | mg | Serine | 700 | mg |
| Aspartic acid | 850 | mg | ***Leucine | 1115 | mg | ***Threonine | 510 | mg |
| Cystine | 65 | mg | ***Lysine | 920 | mg | ***Tryptophan | 145 | mg |
| Glutamic acid | 2640 | mg | ***Methionine | 310 | mg | Tyrosine | 630 | mg |
| Glycine | 250 | mg | ***Phenylalanine | 600 | mg | ***Valine | 755 | mg |

Ingredients:
Fructose, Nonfat Milk Solids, Calcium Sodium Caseinate, Natural and Artificial flavors, Cellulose, Corn Bran, Potassium Chloride, Lecithin, Maltodextrin, Carrageenan, Wheat Germ Oil, (source of Octacosanol), Magnesium Oxide, Beta Carotene, Selenium, Ascorbic Acid, Ferrous Fumarate, d'Alpha Tocopherol Acetate, Chromium, Niacin, Aloe, Apple Pectin, Zinc Oxide, Manganese Sulfate, Vitamin A Palmitate, d-Calcium Pantothenate, Copper Sulfate, Pyridoxine Hydrochloride, Riboflavin, Thiamine Hydrochloride, Cobalamin Concentrate, Vitamin D₂, Folic Acid, Biotin and Potassium Chloride.

42. The method of claim 41, wherein the polymannan extract is administered intravenously at a dose of 10-15 mg three times a week.

43. The method of claim 41, wherein the formulations other than the polymannan extract are administered orally.

44. The method of claim 41, wherein the aloe anthraquinones are administered at a dose ranging from 50-75 mg/day.

45. The method of claim 41, wherein the freeze dried aloe powder, the aloe juice, the aloe gel or the combination is administered at a dose of 400 mg one or more times a day.

46. The method of claim 41, wherein the composition releases one or more cytocommunicators selected from the group consisting of TNF-α, IL-1β, IL-6, and INF-γ, wherein the release stimulates the growth and cytolytic action of one or more Natural Killer (NK) cells in the subject.
